(19) United States
(12) Patent Application Publication
(10) Pub. No.: US 2026/0137509 A1
(43) Pub. Date: May 21, 2026

(54) PROSTHETIC VALVE DOCKING DEVICE

(71) Applicant: Edwards Lifesciences Corporation, Irvine, CA (US)

(72) Inventors: Jocelyn Chau, Buena Park, CA (US); Darshin S. Patel, Lake Forest, CA (US); Tram Ngoc Nguyen, Santa Ana, CA (US)

(21) Appl. No.: 19/450,514

(22) Filed: Jan. 15, 2026

Related U.S. Application Data
(63) Continuation of application No. PCT/US2024/037038, filed on Jul. 8, 2024.
(60) Provisional application No. 63/514,554, filed on Jul. 19, 2023.

(51) Int. Cl. A61F 2/24

(52) U.S. Cl. CPC A61F 2/2412; A61F 2/246; A61F 2220/0008; A61F 2220/0075; A61F 2220/0091; A61F 2230/0004; A61F 2230/0091; A61F 2250/0069

(57) ABSTRACT

A docking device for securing a prosthetic valve at a native valve includes a coil having a plurality of helical turns when deployed at the native valve, and a guard member attached to the coil. The guard member is movable between a radially compressed state and a radially expanded state. The guard member includes a plurality of petals. Each petal includes a rounded head portion and a tapered base portion. The head portion is wider than the base portion when the guard member is in the radially expanded state.

### CROSS-REFERENCE TO RELATED APPLICATIONS

This application is a continuation of PCT Patent Application No. PCT/US 2024/037038 filed on Jul. 8, 2024, which claims the benefit of U.S. Provisional Application No. 63/514,554, filed Jul. 19, 2023, each of these applications being incorporated by reference herein in its entirety.

### FIELD

The present disclosure concerns examples of a docking device configured to secure a prosthetic valve at a native heart valve, as well as methods of assembling such devices.

### BACKGROUND

Prosthetic valves can be used to treat cardiac valvular disorders. Native heart valves (for example, the aortic, pulmonary, tricuspid and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (for example, a native aortic valve). As such, the THV may not be suitable for implantation at another native valve (for example, a native mitral valve) and/or in a patient with a larger native valve. Additionally, or alternatively, the native tissue at the implantation site may not provide sufficient structure for the THV to be secured in place relative to the native tissue. Accordingly, improvements to THVs and the associated transcatheter delivery apparatus are desirable.

### SUMMARY

The present disclosure relates to methods and devices for treating valvular regurgitation and/or other valve issues. Specifically, the present disclosure is directed to a docking device configured to receive a prosthetic valve and the methods of assembling the docking device and implanting the docking device.

A docking device for securing a prosthetic valve at a native valve can include a coil comprising a plurality of helical turns when deployed at the native valve. In addition to these features, a docking device can further comprise one or more of the components disclosed herein.

In some examples, a docking device can include a guard member attached to the coil and is movable between a radially compressed state and a radially expanded state.

In some examples, a guard member can include a plurality of petals.

In some examples, each petal can have a rounded head portion and a tapered base portion, and the head portion is wider than the base portion when the guard member is in the radially expanded state.

In some examples, a guard member can include a wireframe including a spine and a plurality of lobes connected to the spine, the lobes extending radially outwardly from the spine.

Certain aspects of the disclosure concern a guard member for a docking device configured to secure a prosthetic valve at a native valve. The guard member can include a wireframe and a cover substantially enclosing the wireframe. The wireframe can include a spine and a plurality of lobes connected to the spine. The lobes can extend radially outwardly from the spine. The plurality of lobes can be radially expandable and compressible.

Certain aspects of the disclosure concern a method for making a docking device configured to secure a prosthetic valve at a native valve. The method can include obtaining a wireframe comprising a spine and a plurality of lobes connected to and extending radially outwardly from the spine, and enclosing the wireframe with a cover to form a guard member of the docking device.

Certain aspects of the disclosure concern a method for implanting a prosthetic valve. The method includes delivering a docking device to a native valve, deploying the docking device at an annulus of the native valve, and deploying a prosthetic valve within the docking device. The docking device includes a coil and a guard member attached to the coil. The coil can remain in a substantially straight configuration when delivering the docking device and move to a helical configuration after the docking device is deployed. The guard member can remain in a folded configuration when delivering the docking device and move to an unfolded configuration after the docking device is deployed.

The above method can be performed on a living animal or on a simulation, such as on a cadaver, cadaver heart, anthropomorphic ghost, simulator (for example, with body parts, heart, tissue, etc. being simulated).

In some examples, a docking device or a guard member comprises one or more of the components recited in Examples 1-60 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 schematically illustrates a first stage in an exemplary mitral valve replacement procedure where a guide catheter and a guidewire are inserted into a vasculature of a patient and navigated through the vasculature and into a heart of the patient, towards a native mitral valve of the heart.

FIG. 2A schematically illustrates a second stage in the exemplary mitral valve replacement procedure where a docking device delivery apparatus extending through the guide catheter is used to deploy a docking device at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is deploy a prosthetic heart valve within the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a perspective view of an example prosthetic heart valve.

FIG. 6A is a side perspective view of a docking device in a deployed configuration, the docking device including a helical coil and a guard member, according to one example.

FIG. 6B is a top view of the docking device of FIG. 6A.

FIG. 6C is a transverse cross-sectional view of a docking device of FIG. 6A taking along the line 6C-6C.

FIG. 6D is another transverse cross-sectional view of a docking device of FIG. 6A taking along the line 6D-6D.

FIG. 7A depicts a guard member in a radially expanded state, according to one example.

FIG. 7B depicts the guard member of FIG. 7A in a substantially straight configuration.

FIG. 7C depicts the guard member of FIG. 7B being radially compressed.

FIG. 7D depicts the guard member of FIG. 7C in a folded configuration.

FIG. 8A schematically depicts a docking device deployed at a native valve, the docking device comprising another guard member, according to one example.

FIG. 8B schematically depicts a prosthetic valve deployed within the docking device of FIG. 8A, according to one example.

FIG. 9A depicts a wireframe of the guard member of FIG. 8A in an unfolded configuration, according to one example.

FIG. 9B depicts the wireframe of FIG. 9A in a folded configuration.

FIG. 10A depicts a method of cutting two fabric layers using a mold to create a cover for a guard member of a docking device, according to one example.

FIG. 10B depicts a guard member including the cover created according to FIG. 10A and a wireframe enclosed within cover.

FIG. 10C depicts attaching the wireframe to the cover of the guard member of FIG. 10B, according to one example.

FIG. 10D depicts attaching the guard member of FIG. 10C to a coil of a docking device, according to one example.

### DETAILED DESCRIPTION

### General Considerations

It should be understood that the disclosed examples can be adapted to deliver and implant prosthetic devices in any of the native annuluses of the heart (for example, the pulmonary, mitral, and tricuspid annuluses), and can be used with any of various delivery approaches (for example, retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (for example, mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Exemplary Transcatheter Heart Valve Replacement Procedure

Described herein are various systems, apparatuses, methods, or the like, that can be used in or with delivery apparatuses to deliver a prosthetic implant (for example, a prosthetic valve, a docking device, etc.) into a patient body.

In certain examples, a delivery apparatus can be configured to deliver and implant a docking device at an implantation site, such as a native valve annulus. The docking device can be configured to more securely hold an expandable prosthetic valve implanted within the docking device, at the native valve annulus. For example, a docking device can provide or form a more circular and/or stable anchoring site, landing zone, or implantation zone at the implant site, in which a prosthetic valve can be expanded or otherwise implanted. By providing such anchoring or docking devices, replacement prosthetic valves can be more securely implanted and held at various valve annuluses, including at the mitral annulus which does not have a naturally circular cross-section.

In some examples, the docking device can be arranged within an outer shaft of the delivery apparatus. A sleeve shaft can cover or surround the docking device within the delivery apparatus and during delivery to a target implantation site. A pusher shaft can be disposed within the outer shaft, proximal to the docking device, and configured to push the docking device out of the outer shaft to position the docking device at the target implantation site. The sleeve shaft can also surround the pusher shaft within the outer shaft of the delivery apparatus. After positioning the docking device at the target implantation site, the sleeve shaft can be removed from the docking device and retracted back into the outer shaft of the delivery apparatus.

Fluid (for example, a flush fluid, such as heparinized saline or the like) can be provided to a pusher shaft lumen defined within an interior of the pusher shaft, a delivery shaft lumen defined between the sleeve shaft and the outer shaft of the delivery apparatus, and a sleeve shaft lumen defined between the pusher shaft and the sleeve shaft. By providing a consistent flow of fluid through these lumens of the delivery apparatus, stagnation of blood within the delivery apparatus can be reduced or avoided, thereby reducing a risk of thrombus formation.

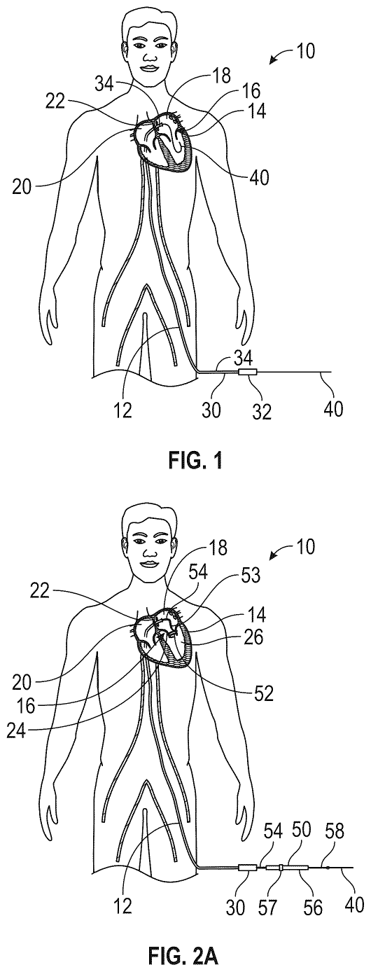

An exemplary transcatheter heart valve replacement procedure which utilizes a first delivery apparatus to deliver a docking device to a native valve annulus and then a second delivery apparatus to deliver a prosthetic heart valve (for example, THV) inside the docking device is depicted in the schematic illustrations of FIGS. 1-4.

As introduced above, defective native heart valves may be replaced with THVs. However, in certain instances, such THVs may not be able to sufficiently secure themselves to the native tissue (for example, to the leaflets and/or annulus of the native heart valve) and may undesirably shift around relative to the native tissue, leading to paravalvular leakage, valve malfunction, and/or other issues. Thus, a docking device may be implanted first at the native valve annulus and then the THV can be implanted within the docking device to help anchor the THV to the native tissue and provide a seal between the native tissue and the THV.

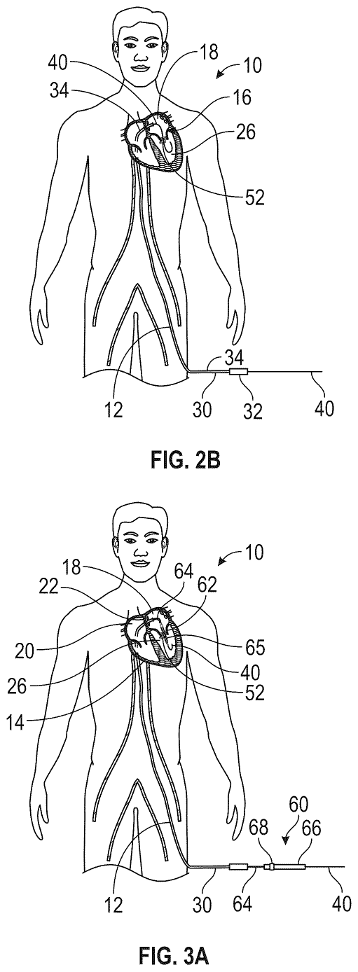

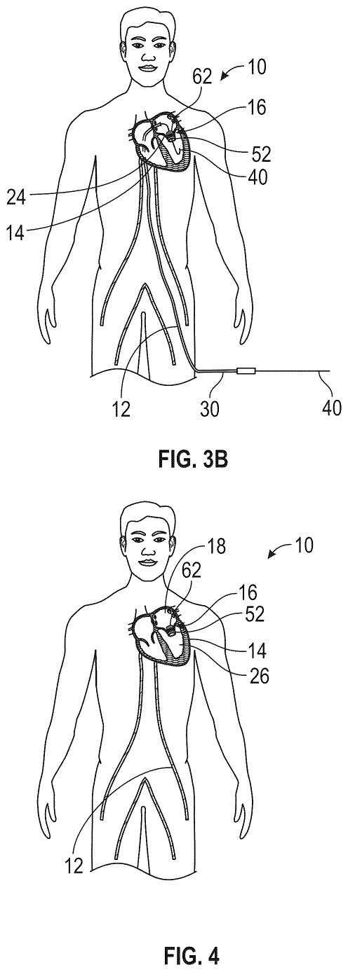

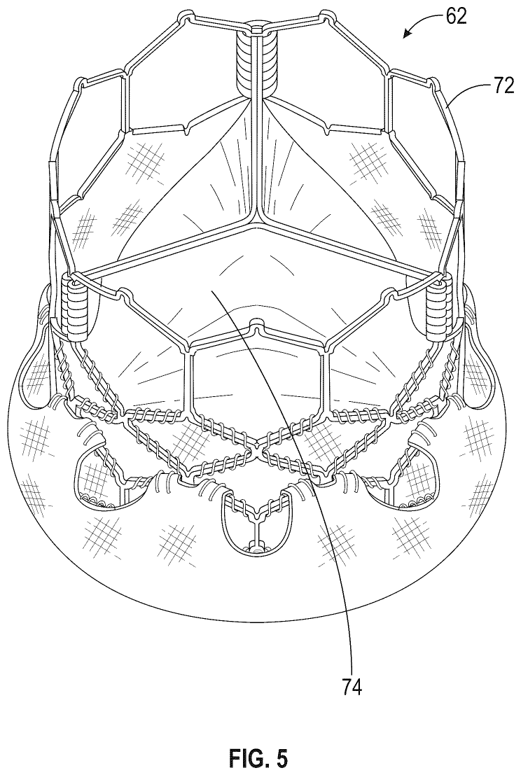

FIGS. 1-4 depict an exemplary transcatheter heart valve replacement procedure (for example, a mitral valve replacement procedure) which utilizes a docking device 52 and a prosthetic heart valve 62, according to one example. During the procedure, a user can create a pathway to a patient's native heart valve using a guide catheter 30 (FIG. 1). The user can deliver and implant the docking device 52 at the patient's native heart valve using a docking device delivery apparatus 50 (FIG. 2A) and then removes the docking device delivery apparatus 50 from the patient 10 after implanting the docking device 52 (FIG. 2B). The user can then implant the prosthetic heart valve 62 within the implanted docking device 52 using a prosthetic valve delivery apparatus 60 (FIG. 3A). Thereafter, the user can remove the prosthetic valve delivery apparatus 60 from the patient 10 (FIG. 3B), as well as the guide catheter 30 (FIG. 4).

FIG. 1 depicts a first stage in a mitral valve replacement procedure, according to one example. As shown, the guide catheter 30 and a guidewire 40 can be inserted into a vasculature 12 of a patient 10 and navigated through the vasculature 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (for example, the native mitral valve 16 or native mitral valve annulus).

Initially, the user may first make an incision in the patient's body to access the vasculature 12. For example, as illustrated in FIG. 1, the user may make an incision in the patient's groin to access a femoral vein. Thus, in such examples, the vasculature 12 may include a femoral vein.

After making the incision to access the vasculature 12, the user may insert the guide catheter 30, the guidewire 40, and/or additional devices (such as an introducer device or transseptal puncture device) through the incision and into the vasculature 12. The guide catheter 30 (which can also be referred to as an “introducer device,” “introducer,” or “guide sheath”) can be configured to facilitate the percutaneous introduction of various implant delivery devices (for example, the docking device delivery apparatus 50 and the prosthetic valve delivery apparatus 60) into and through the vasculature 12 and may extend through the vasculature 12 and into the heart 14 but may stop short of the native mitral valve 16. The guide catheter 30 can comprise a handle 32 and a shaft 34 extending distally from the handle 32. The shaft 34 can extend through the vasculature 12 and into the heart 14 while the handle 32 can remain outside the body of the patient 10 and can be operated by the user in order to manipulate the shaft 34 (FIG. 1).

The guidewire 40 can be configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the vasculature 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to access the vasculature 12, the user may insert a transseptal puncture device through the incision and into the vasculature 12. The user may guide the transseptal puncture device through the vasculature 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the vasculature 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the vasculature 12 and advance the guide catheter 30 into the left atrium 18 over the guidewire 40 (FIG. 1).

In some instances, an introducer device can be inserted through a lumen of the guide catheter 30 prior to inserting the guide catheter 30 into the vasculature 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 can be implanted at the native mitral valve 16 of the heart 14 of the patient 10 using a docking device delivery apparatus 50 (which may also be referred to as an “implant catheter,” or a “docking device delivery device,” or simply “delivery apparatus”).

In general, the docking device delivery apparatus 50 can include a delivery shaft 54 (which may also be referred to as an “outer shaft”), a handle 56, and a pusher assembly 58 (which may also be referred to as a “pusher shaft”). The delivery shaft 54 can be configured to be advanced through the patient's vasculature 12 and to the implantation site (for example, native mitral valve 16) by the user, and may be configured to retain the docking device 52 in a distal end portion 53 of the delivery shaft 54. In some examples, the distal end portion 53 of the delivery shaft 54 can retain the docking device 52 therein in a substantially straight delivery configuration.

The handle 56 of the docking device delivery apparatus 50 can be configured to be gripped and/or otherwise held by the user to advance the delivery shaft 54 through the patient's vasculature 12. Specifically, the handle 56 can be coupled to a proximal end of the delivery shaft 54 and can be configured to remain accessible to the user (for example, outside the body of the patient 10) during the docking device implantation procedure. In this way, the user can advance the delivery shaft 54 through the patient's vasculature 12 by exerting a force on (for example, pushing) the handle 56. In some examples, the delivery shaft 54 can be configured to carry the pusher assembly 58 and/or the docking device 52 with it as it advances through the patient's vasculature 12. In this way, the docking device 52 and/or the pusher assembly 58 can advance through the patient's vasculature 12 in lockstep with the delivery shaft 54 as the user grips the handle 56 and pushes the delivery shaft 54 deeper into the patient's vasculature 12.

In some examples, the handle 56 can comprise one or more articulation members 57 that are configured to aid in navigating the delivery shaft 54 through the vasculature 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the vasculature 12 and/or within the heart 14.

The pusher assembly 58 can be configured to deploy and/or implant the docking device 52 at the implantation site (for example, the native mitral valve 16). For example, the pusher assembly 58 can be configured to be adjusted by the user to push the docking device 52 out of the distal end portion 53 of the delivery shaft 54. A pusher shaft of the pusher assembly 58 can extend through the delivery shaft 54 and can be disposed adjacent to the docking device 52 within the delivery shaft 54. In some examples, the docking device 52 can be releasably coupled to the pusher shaft of the pusher assembly 58 via a connection mechanism of the docking device delivery apparatus 50 such that the docking device 52 can be released after being deployed at the native mitral valve 16. Because the docking device 52 is retained by, held, and/or otherwise coupled to the pusher assembly 58, the docking device 52 can advance in lockstep with the pusher assembly 58 through and/or out of the delivery shaft 54.

In addition to the pusher shaft, in certain instances, the pusher assembly 58 can also include a sleeve shaft. The pusher shaft can be configured to advance the docking device 52 through the delivery shaft 54 and out of the distal end portion 53 of the delivery shaft 54, while the sleeve shaft, when included, can have a distal dock sleeve configured to cover the docking device 52 within the delivery shaft 54 and while pushing the docking device 52 out of the delivery shaft 54 and positioning the docking device 52 at the implantation site. In some examples, the pusher shaft can be covered, at least in part, by the sleeve shaft.

In some examples, the pusher assembly 58 can comprise a pusher handle that is coupled to the pusher shaft and that is configured to be gripped and pushed by the user to translate the pusher shaft axially relative to the delivery shaft 54 (for example, to push the pusher shaft into and/or out of the distal end portion 53 of the delivery shaft 54). The dock sleeve can be configured to be retracted and/or withdrawn from the docking device 52, after positioning the docking device 52 at the target implantation site. For example, the pusher assembly 58 can include a sleeve handle that is coupled to the sleeve shaft and is configured to be pulled by a user to retract (for example, axially move) the sleeve shaft relative to the pusher shaft, thereby retracting the dock sleeve.

The pusher assembly 58 can be removably coupled to the docking device 52, and as such can be configured to release, detach, decouple, and/or otherwise disconnect from the docking device 52 once the docking device 52 has been deployed at the target implantation site. As just one example, the pusher assembly 58 may be removably coupled to the docking device 52 via a thread, string, yarn, suture, or other suitable material that is tied or sutured to the docking device 52.

In some examples, the pusher assembly 58 can include a suture lock assembly (also referred to as a “suture lock”) that is configured to receive and/or hold the thread or other suitable material that is coupled to the docking device 52 via a suture. The thread or other suitable material that forms the suture can extend from the docking device 52, through the pusher assembly 58, to the suture lock assembly. The suture lock assembly can also be configured to cut the suture to release, detach, decouple, and/or otherwise disconnect the docking device 52 from the pusher assembly 58. For example, the suture lock assembly can comprise a cutting mechanism that is configured to be adjusted by the user to cut the suture.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the vasculature 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the vasculature 12 and the heart 14, the user may adjust the one or more articulation members 57 of the handle 56 to navigate the various turns, corners, constrictions, and/or other obstacles in the vasculature 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the medial commissure of the native mitral valve 16. For example, the user can maintain the position of the pusher assembly 58 (for example, by exerting a holding and/or pushing force on the pusher shaft) while retracting the delivery shaft 54 proximally so that the delivery shaft 54 withdraws and/or otherwise retracts relative to the docking device 52 and the pusher assembly 58. In this way, the pusher assembly 58 can hold the docking device 52 in place while the user retracts the delivery shaft 54, thereby releasing the docking device 52 from the delivery shaft 54. In some examples, the user can also remove the dock sleeve from the docking device 52, for example, by retracting the sleeve shaft.

After deploying and implanting the docking device 52 at the native mitral valve 16, the user may disconnect the docking device delivery apparatus 50 from the docking device 52. Once the docking device 52 is disconnected from the docking device delivery apparatus 50 (for example, by cutting the suture tied to the docking device 52), the user may retract the docking device delivery apparatus 50 out of the vasculature 12 and away from the patient 10 so that the user can deliver and implant a prosthetic heart valve 62 within the implanted docking device 52 at the native mitral valve 16.

FIG. 2B depicts a third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of helical turns that wrap around the leaflets 24 of the native mitral valve 16 (within the left ventricle 26). The implanted docking device 52 can have a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 within the docking device 52 using a prosthetic valve delivery apparatus 60.

As shown in FIG. 3A, the prosthetic valve delivery apparatus 60 can comprise a delivery shaft 64 and a handle 66. The delivery shaft 64 can extend distally from the handle 66. The delivery shaft 64 can be configured to extend into the patient's vasculature 12 to deliver, implant, expand, and/or otherwise deploy the prosthetic heart valve 62 within the docking device 52 at the native mitral valve 16. The handle 66 can be configured to be gripped and/or otherwise held by the user to advance the delivery shaft 64 through the patient's vasculature 12.

In some examples, the handle 66 can comprise one or more articulation members 68 that are configured to aid in navigating the delivery shaft 64 through the vasculature 12 and the heart 14. Specifically, the articulation members 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the vasculature 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 can be mounted around the expansion mechanism 65 (for example, the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (for example, the delivery shaft 64) into the patient 10 through the guide catheter 30 and over the guidewire 40. The user can continue to advance the prosthetic valve delivery apparatus 60 along the guidewire 40 (for example, through the vasculature 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, pushing) the handle 66. While advancing the delivery shaft 64 through the vasculature 12 and the heart 14, the user can adjust the one or more articulation members 68 of the handle 66 to navigate the various turns, corners, constrictions, and/or other obstacles in the vasculature 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52. In some examples, the user can lock the prosthetic heart valve 62 in its fully expanded position (for example, with a locking mechanism) to prevent the prosthetic heart valve 62 from collapsing.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 can be received and retained within the docking device 52.

As also shown in FIG. 3B, after the prosthetic heart valve 62 has been fully deployed and implanted within the docking device 52 at the native mitral valve 16, the prosthetic valve delivery apparatus 60 (including the delivery shaft 64) can be removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10. The docking device 52 can be configured to provide a seal between the prosthetic heart valve 62 and the leaflets 24 of the native mitral valve 16 to reduce paravalvular leakage around the prosthetic heart valve 62. Specifically, the docking device 52 can initially constrict the leaflets 24 of the native mitral valve 16. The prosthetic heart valve 62 can then push the leaflets 24 against the docking device 52 as it radially expands within the docking device 52. Thus, the docking device 52 and the prosthetic heart valve 62 can be configured to sandwich the leaflets 24 of the native mitral valve 16 when the prosthetic heart valve 62 is expanded within the docking device 52. In this way, the docking device 52 can provide a seal between the leaflets 24 of the native mitral valve 16 and the prosthetic heart valve 62 to reduce paravalvular leakage around the prosthetic heart valve 62.

In some examples, one or more of the docking device delivery apparatus 50, the prosthetic valve delivery apparatus 60, and/or the guide catheter 30 can comprise one or more fluid ports that are configured to supply flushing fluid to the lumens thereof to prevent and/or reduce the likelihood of blood clot (for example, thrombus) formation. Example fluid ports that can be used to inject flushing fluid into a docking device delivery apparatus are described further below.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned or disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

Additional examples of the docking device delivery apparatus, including its variants, and methods of implanting a docking device and implanting a prosthetic valve within the docking device are described in International Publication Nos. WO 2020/247907 and WO 2022/087336, and U.S. Patent Publication Nos. US 2018/0318079, US 2018/0263764, and US2018/0177594, which are all incorporated by reference herein in their entireties.

### Exemplary Prosthetic Valves

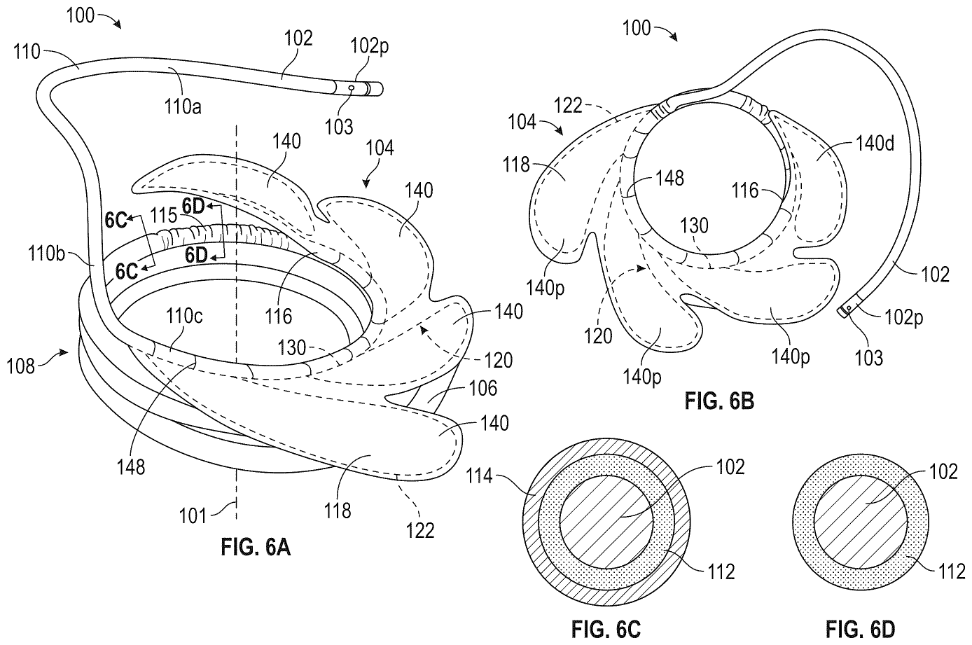

FIG. 5 is a perspective view of the prosthetic heart valve 62, according to one example. As shown, the heart valve 62 comprises a frame, or stent, 72 and a leaflet structure 74 supported by the frame. In some examples, the prosthetic heart valve 62 is adapted to be implanted in the native aortic valve and can be implanted in the body using, for example, the prosthetic valve delivery apparatus 60 described above.

In some examples, the frame 72 comprises a plastically expandable material, which can be metal alloys, polymers, or combinations thereof. Example metal alloys can comprise one or more of the following: nickel, cobalt, chromium, molybdenum, titanium, or other biocompatible metal. In some examples, the frame 72 can comprise stainless steel. In some examples, the frame 72 can comprise cobalt-chromium. In some examples, the frame 72 can comprise nickel-cobalt-chromium. In some examples, the frame 72 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

In some examples, the prosthetic valve 62 can be a self-expandable prosthetic valve with a frame made from a self-expanding material, such as nickel titanium or Nitinol. When the prosthetic valve is a self-expanding valve, the balloon of the delivery apparatus can be replaced with a sheath or similar restraining device that retains the prosthetic valve in a radially compressed state for delivery through the body. When the prosthetic valve is at the implantation location, the prosthetic valve can be released from the sheath, and therefore allowed to expand to its functional size. It should be noted that any of the delivery apparatuses disclosed herein can be adapted for use with a self-expanding valve.

Additional details regarding the prosthetic valves described herein and various valve components are described U.S. Pat. No. 11,185,406, which is incorporated herein by reference. Additional example prosthetic valves are described in International Patent Application Publication No. WO 2018/222799, U.S. Pat. No. 9,155,619, and U.S. Patent Publication No. 2018/0028310, all of which are incorporated herein by reference in their entireties.

### Overview of Docking Devices

Docking devices according to examples of the disclosure can, for example, provide a stable anchoring site, landing zone, or implantation zone at the implant site in which prosthetic valves can be expanded or otherwise implanted. Many of the disclosed docking devices comprise a circular or cylindrically-shaped portion, which can (for example) allow a prosthetic heart valve comprising a circular or cylindrically-shaped valve frame to be expanded or otherwise implanted into native locations with naturally circular cross-sectional profiles and/or in native locations with naturally with non-circular cross sections. In addition to providing an anchoring site for the prosthetic valve, the docking devices can be sized and shaped to cinch or draw the native valve (for example, mitral, tricuspid, etc.) anatomy radially inwards. In this manner, one of the main causes of valve regurgitation (for example, functional mitral regurgitation), specifically enlargement of the heart (for example, enlargement of the left ventricle, etc.) and/or valve annulus, and consequent stretching out of the native valve (for example, mitral, etc.) annulus, can be at least partially offset or counteracted. Some examples of the docking devices further include features which, for example, are shaped and/or modified to better hold a position or shape of the docking device during and/or after expansion of a prosthetic valve therein. By providing such docking devices, replacement valves can be more securely implanted and held at various valve annuluses, including at the mitral valve annulus which does not have a naturally circular cross-section.

In some instances, a docking device can comprise a paravalvular leakage (PVL) guard (also referred to herein as “a guard member”). The PVL guard can, for example, help reduce regurgitation and/or promote tissue ingrowth between the native tissue and the docking device.

The PVL guard can, in some examples, be movable between a delivery configuration (or radially compressed state) and a deployed configuration (or radially expanded state). When the PVL guard is in the delivery configuration, the PVL guard can extend along and adjacent the coil. When the PVL guard is in the deployed configuration, the PVL guard can rotate about a central longitudinal axis of the coil and extend radially outwardly from the coil.

### Exemplary Docking Devices

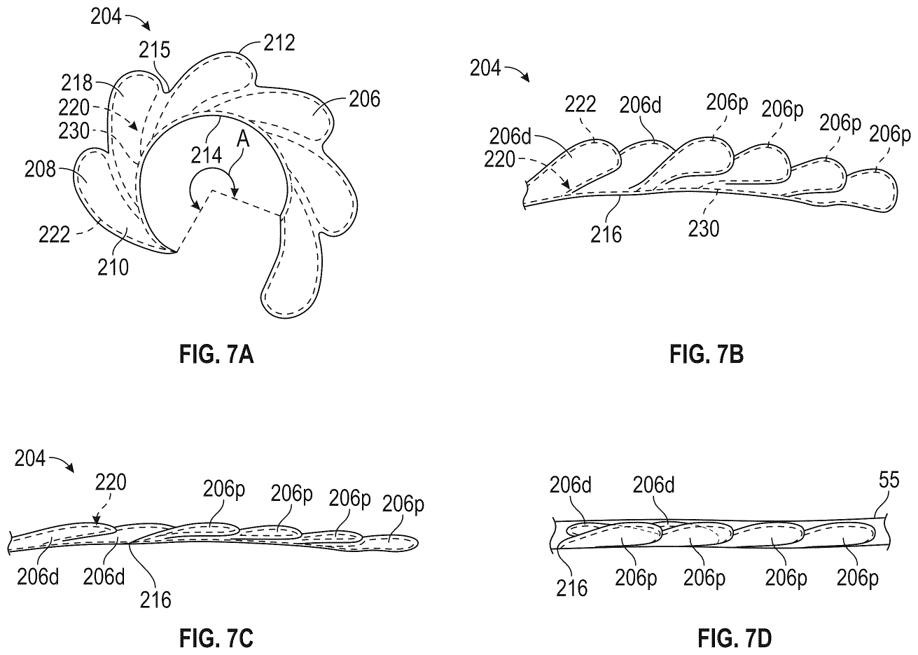

FIGS. 6A-6D show a docking device 100, according to one example. The docking device 100 can, for example, be implanted within a native valve annulus. The docking device 100 can be configured to receive and secure a prosthetic valve (for example, prosthetic heart valve 62), thereby securing the prosthetic valve at the native valve annulus.

The docking device 100 can comprise a coil 102 and a guard member 104 (which can also be referred to as “a PVL guard” or “a sealing member”) extending along at least a portion of the coil 102. In certain examples, the coil 102 can include a shape memory material (for example, nickel titanium alloy or Nitinol) such that the docking device 100 (and the coil 102) can move from a substantially straight configuration (also referred to as “delivery configuration”) when disposed within a delivery sheath of a delivery apparatus (for example, docking device delivery apparatus 50) to a helical configuration (also referred to as “deployed configuration,” as shown in FIG. 6A) after being removed from the delivery sheath.

During delivery of the docking device and after initial deployment of the docking device at the implantation site, the guard member 104 can be retained in a radially compressed state by a dock sleeve of the delivery apparatus. After the docking device 100 is deployed at the implantation site, the dock sleeve can be removed so as to expose the guard member 104, thereby allowing the guard member 104 to move to a radially expanded state.

In some examples, when the docking device 100 is in the deployed configuration and the guard member 104 is in the radially expanded state, the guard member 104 can extend circumferentially relative to a central longitudinal axis 101 of the docking device 100 from 180 degrees to 400 degrees, or from 210 degrees to 330 degrees, or from 250 degrees to 290 degrees, or from 260 degrees to 280 degrees (for example, 270 degrees) relative to the central longitudinal axis 101. In other words, the guard member 104 can extend circumferentially from about one half of a revolution (for example, 180 degrees) around the central longitudinal axis 101 in some examples to more than a full revolution (for example, 400 degrees) around the central longitudinal axis 101 in other examples, including various ranges in between. As used herein, a range (for example, from 180 degrees to 400 degrees, and between 180 degrees and 400 degrees) includes the endpoints of the range (for example, 180 degrees and 400 degrees).

The coil 102 has a proximal end 102p and a distal end, which also respectively define the proximal and distal ends of the docking device 100. When being disposed within the delivery sheath (for example, during delivery of the docking device into the vasculature of a patient), a body of the coil 102 between the proximal end 102p and distal end can form the generally straight delivery configuration (that is, without any coiled or looped portions, but can be flexed or bent) so as to maintain a small radial profile when moving through a patient's vasculature. After being removed from the delivery sheath and deployed at an implant position, the coil 102 can move from the delivery configuration to the helical deployed configuration and wrap around native tissue adjacent the implant position. For example, when implanting the docking device at the location of a native valve, the coil 102 can be configured to surround native leaflets of the native valve (and the chordae tendineae that connects native leaflets to adjacent papillary muscles, if present).

The docking device 100 can be releasably coupled to a delivery apparatus (for example, docking device delivery apparatus 50). For example, in certain examples, the docking device 100 can be coupled to the delivery apparatus via a release suture that can be configured to be tied to the docking device 100 and cut for removal. In one example, the release suture can be tied to the docking device 100 through an eyelet or eyehole 103 located adjacent the proximal end 102p of the coil. In another example, the release suture can be tied around a circumferential recess that is located adjacent the proximal end 102p of the coil 102.

In some examples, the docking device 100 in the deployed configuration can be configured to fit at the mitral valve position. In other examples, the docking device 100 can also be shaped and/or adapted for implantation at other native valve positions as well, such as at the tricuspid valve. As described herein, the geometry of the docking device 100 can be configured to engage the native anatomy, which can, for example, provide for increased stability and reduction of relative motion between the docking device 100, the prosthetic valve docked therein, and/or the native anatomy. Reduction of such relative motion can, among other things, prevent material degradation of components of the docking device 100 and/or the prosthetic valve docked therein and/or prevent damage or trauma to the native tissue.

As shown in FIG. 6A, the coil 102 in the deployed configuration can include a leading turn 106 (or “leading coil”), a central region 108, and a stabilization turn 110 (or “stabilization coil”) around the central longitudinal axis 101. The central region 108 can possess one or more helical turns having substantially equal inner diameters. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations).

In some examples, the central region 108 can include a plurality of helical turns (for example, the docking device 100 can have three helical turns in the central region 108). Some of the helical turns in the central region 108 can be full turns (that is, rotating 360 degrees). In some examples, the most proximal turn and/or the most distal turn can be partial turns (for example, rotating less than 360 degrees, such as 180 degrees, 270 degrees, etc.).

The size of the docking device 100 can be generally selected based on the size of the desired prosthetic valve to be implanted into the patient. In some examples, the central region 108 can be configured to retain a radially expandable prosthetic valve. For example, the inner diameter of the helical turns in the central region 108 can be configured to be smaller than an outer diameter of the prosthetic valve when the prosthetic valve is radially expanded so that additional radial force can act between the central region 108 and the prosthetic valve to hold the prosthetic valve in place. The helical turns in the central region 108 can also be referred to herein as “functional turns.”

The stabilization turn 110 can be configured to help stabilize the docking device 100 in the desired position. For example, the radial dimension of the stabilization turn 110 can be significantly larger than the radial dimension of the coil in the central region 108, so that the stabilization turn 110 can flare or extend sufficiently outwardly so as to abut or push against the walls of the circulatory system, thereby improving the ability of the docking device 100 to stay in its desired position prior to the implantation of the prosthetic valve. In some examples, the diameter of stabilization turn 110 is desirably larger than the native annulus, native valve plane, and/or native chamber for better stabilization. In some examples, the stabilization turn 110 can be a full turn (that is, rotating about 360 degrees). In some examples, the stabilization turn 110 can be a partial turn (for example, rotating between about 180 degrees and about 270 degrees).

In one particularly example, when implanting the docking device 100 at the native mitral valve location, the functional turns in the central region 108 can be disposed substantially in the left ventricle and the stabilization turn 110 can be disposed substantially in the left atrium. The stabilization turn 110 can be configured to provide one or more points or regions of contact between the docking device 100 and the left atrial wall, such as at least three points of contact in the left atrium or complete contact on the left atrial wall. In some examples, the points of contact between the docking device 100 and the left atrial wall can form a plane that is approximately parallel to a plane of the native mitral valve.

In some examples, the stabilization turn 110 can have an atrial portion 110c (attached to the guard member 104 in FIG. 6A) in connection with the central region 108, a stabilization portion 110a adjacent to the proximal end 102p of the coil 102, and an ascending portion 110b located between the atrial portion 110c and the stabilization portion 110a. Both the atrial portion 110c and the stabilization portion 110a can be generally parallel to the helical turns in the central region 108, whereas the ascending portion 110b can be oriented to be angular relative to the atrial portion 110c and the stabilization portion 110a. For example, in certain examples, the ascending portion 110b and the stabilization portion 110a can form an angle from about 45 degrees to about 90 degrees (inclusive). When implanting the docking device 100 at the native mitral valve location, the atrial portion 110c can be configured to abut a posterior wall of the left atrium and the stabilization portion 110a can be configured to flare out and press against an anterior wall of the left atrium.

As noted above, the leading turn 106 can have a larger radial dimension than the helical turns in the central region 108. The leading turn 106 can help more easily guide the coil 102 around and/or through the chordae tendineae and/or adequately around all native leaflets of the native valve (for example, the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (that is, rotating about 360 degrees). In some examples, the leading turn 106 can be a partial turn (for example, rotating between about 180 degrees and about 270 degrees). When a prosthetic valve is radially expanded within the central region 108 of the coil, the functional turns in the central region 108 can be further radially expanded. As a result, the leading turn 106 can be pulled in the proximal direction and become a part of the functional turn in the central region 108.

In some examples, at least a portion of the coil 102 can be at least partially surrounded by a cover. The cover can, for example, prevent or reduce trauma to native tissue and/or prevent or reduce damage to the delivery device, reduce friction with the native tissue, increase friction with the native tissue and/or prosthetic heart valve, etc. In some instances, the coil can comprise a plurality of covers and/or a plurality of sections of one or more covers, each configured for a particular purpose. For example, a first cover can be provided over all or at least substantially all of the coil, for example, to prevent or reduce trauma to the native tissue. A second cover can extend over a portion of the first cover and can, for example, be configured to increase friction between the cover and native leaflet tissue. Additional information about the covers is provided below and can be found in International Publication No. WO 2022/087336.

As shown in FIGS. 6C-6D, at least a portion of the coil 102 can be surrounded by an inner cover 112 (which can also be referred to as “a first cover”). The inner cover 112 can have a tubular shape. In some examples, the inner cover 112 can cover an entire length of the coil 102. In some examples, the inner cover 112 covers only selected portion(s) of the coil 102.

In some examples, the inner cover 112 can be coated on and/or bonded on the coil 102. In some examples, the inner cover 112 can be a cushioned, padded-type layer protecting the coil 102. The inner cover 112 can be constructed of various natural and/or synthetic materials. In one particularly example, the inner cover 112 can include a foam material (e.g., expanded polytetrafluoroethylene (ePTFE)). In some examples, the inner cover 112 is configured to be fixedly attached to the coil 102 (for example, by means of textured surface resistance, suture, glue, thermal bonding, or any other means) so that relative axial movement between the inner cover 112 and the coil 102 is restricted or prohibited. In some examples, one or more portions of the inner cover (e.g., a distal end portion) can be fixedly attached to the coil and one or more other portions of the inner cover (e.g., an intermediate portion and/or a proximal end portion) can be movable relative to the coil.

In some examples, as shown in FIG. 6C, the docking device 100 can also include a retention member 114 (which may also be referred to as “a second cover” or “an outer cover”) surrounding at least a portion of the inner cover 112 (and the coil 102). In some examples, retention member 114 can extend over the entire length of the inner cover 112. In the illustrated example, the retention member 114 extends over only a portion of the inner cover 112 so that one or more portions of the inner cover 112 (e.g., the proximal and/or distal end portions) are exposed. In particular examples, a proximal end of the retention member 114 can be positioned proximal to a proximal end of the guard member 104. For example, the proximal end of the retention member 114 can be disposed at or adjacent the ascending portion 110b of the coil 102. In some examples, a distal end of the retention member 114 can be positioned distal to a distal end of the guard member 104. For example, the distal end of the retention member 114 can be positioned adjacent the leading turn 106. In some examples, the retention member 114 can cover the functional turns of the coil 102 in the central region 108. Thus, when the docking device 100 is deployed at the native valve and the prosthetic valve is radially expanded within the docking device 100, the retention member 114 at the central region 108 can frictionally engage the prosthetic heart valve and/or the native leaflet tissue.

The retention member 114 can be formed of various materials configured to engage the native tissue and/or prosthetic heart valve to increase friction therebetween and/or promote tissue ingrown. For example, the retention member can comprise a biocompatible fabric material (e.g., polyethylene terephthalate (PET)). In some examples, the retention member 114 can comprise a braided material. In some examples, the retention member 114 can include a woven material.

In some examples, the guard member 104 can be fixedly attached to the retention member 114 and/or the inner cover 112, for example, via sutures 148, adhesive, and/or any other suitable means for attaching.

In some examples, the guard member 104 can extend along a portion (for example, the atrial portion) of the stabilization turn 110 of the coil 102. In some examples, the guard member 104 can extend along at least a portion of the central region 108 of the coil 102 (for example, a portion of the most proximal turn). In some examples, the guard member 104 can extend along a majority (or even an entirety) of the functional turns in the central region 108. In one example, when the docking device 100 is deployed at a native atrioventricular valve, the guard member 104 does not extend into the ascending portion 110b.

In various examples, the guard member 104 can move between a radially compressed state and a radially expanded state. Specifically, the guard member 104 can include a plurality of petals 140 which can be radially expandable and compressible. In the example depicted in FIGS. 6A-6B, the guard member 104 has four petals 140, including one distal petal 140d and three proximal petals 140p. In other examples, the guard member 104 can have two, three, or more than four petals 140. The petals 140 can extend circumferentially along a portion of the coil 102 of the docking device 100.

When the guard member 104 is in the radially compressed state, the petals 140 can be radially compressed against the coil 102 so that the radial profile of the docking device 100 is smaller than a predefined threshold, for example, between 2 mm and 3 mm, inclusive. When the guard member 104 moves from the radially compressed state to the radially expanded state, the petals 140 can extend radially outwardly relative to the coil 102. The guard member 104 can be biased toward the radially expanded state. Thus, the guard member 104 can be retained in the radially compressed state by a dock sleeve of a delivery apparatus, and automatically return to the radially expanded state after the dock sleeve is removed.

In some examples, the guard member 104 can be folded at a hinge potion 116 which separates the distal petal 140d from the proximal petals 140p. For example, when the guard member 104 is in the radially compressed state (for example, retained within the dock sleeve), the guard member 104 can remain folded such that the distal petal 140d overlaps with at least one of the proximal petals 140p, as described more fully below. When the guard member 104 is in the radially expanded state (for example, after removing the dock sleeve), the guard member 104 can unfold itself such that the petals 140 can surround the native annulus. To allow folding and unfolding of the guard member 104 at the hinge portion 116, the proximal petals 140p can be fixedly attached to the coil 102 (for example, using sutures) while the distal petal 140d can be detached from the coil 102.

The guard member 104 can include a wireframe 120 and a cover 118 substantially enclosing the wireframe 120. The shape of the wireframe 120 can generally define the shape of the guard member 104. For example, the wireframe 120 can include a spine 130 and a plurality of lobes 122 connected to the spine 130. The spine 130 defines an inner edge of the guard member 104 and can be attached to the coil 102. Each lobe 122 can extend radially outwardly from the spine 130 within a corresponding petal 140.

In some examples, the wireframe 120 can include a shape memory material that is shape set and/or pre-configured to expand the guard member 104 to the radially expanded state when unconstrained (for example, when deployed at a native valve location). For example, the wireframe 120 can contain a shape memory alloy with super-elastic properties, such as Nitinol. In some examples, the wireframe 120 can contain a ternary shape memory alloy with superelastic properties, such as NiTiX where X can be chromium (Cr), cobalt (Co), zirconium (Zr), hafnium (Hf), etc.

In some examples, the wireframe 120 can comprise a metallic material that does not have the shape memory properties. In such circumstances, the wireframe 120 can have a biasing mechanism (for example, using springs, etc.) configured to bias the wireframe 120 (and the guard member 104) to the radially expanded state. Examples of such metallic material include cobalt-chromium, stainless steel, etc. In one specific example, the wireframe 120 can comprise nickel-free austenitic stainless steel in which nickel can be completely replaced by nitrogen. In another specific example, the wireframe 120 can comprise cobalt-chromium or cobalt-nickel-chromium-molybdenum alloy with significantly low density of titanium.

In some examples, the cover 118 can be configured to be so elastic that when the guard member 104 moves from the delivery configuration to the deployed configuration, the cover 118 can accommodate the wireframe 120.

In some examples, the cover 118 can be configured to be atraumatic to native tissue and/or promote tissue ingrowth into the cover 118. For example, the cover 118 can have pores to encourage tissue ingrowth. In another example, the cover 118 can be impregnated with growth factors to stimulate or promote tissue ingrowth, such as transforming growth factor alpha (TGF-alpha), transforming growth factor beta (TGF-beta), basic fibroblast growth factor (bFGF), vascular epithelial growth factor (VEGF), and combinations thereof. The cover 118 can be constructed of any suitable material, including foam, cloth, fabric, and/or polymer, which is flexible to allow for compression and expansion of the cover 118. In one example, the cover 118 can include a fabric layer constructed from a thermoplastic polymer material, such as polyethylene terephthalate (PET).

In some examples, the cover 118 can be configured to engage with the prosthetic valve deployed within the docking device so as to form a seal and reduce paravalvular leakage between the prosthetic valve and the docking device after the guard member 104 is radially expanded. The cover 118 can also be configured to engage with the native tissue (for example, the native annulus and/or native leaflets) to reduce PVL between the docking device and/or the prosthetic valve and the native tissue.

As described herein, radial expansion of the guard member 104 can help preventing and/or reducing paravalvular leakage. Specifically, radial expansion of the guard member 104 can form an improved seal around a prosthetic valve deployed within the docking device 100. In some examples, the guard member 104 can be configured to prevent and/or inhibit leakage at the location where the docking device 100 crosses between leaflets of the native valve (for example, at the commissures of the native leaflets). For example, without the guard member 104, the docking device 100 may push the native leaflets apart at the point of crossing the native leaflets and allow for leakage at that point (for example, along the docking device or to its sides). However, the guard member 104 can be configured to expand to cover and/or fill any opening at that point and inhibit leakage along the docking device 100.

In some examples, the inner cover 112 and/or the retention member 114 can have slack. For example, FIG. 6A shows that the inner cover 112 can be axially compressed to have slack 115 before radially expanding a prosthetic valve within the docking device 100. The inner cover 112 can be constructed with a low density ePTFE so that the inner cover 112 can be axially compressed and the resulting slack 115 does not significantly impact the radial profile of the docking device 100. When radially expanding a prosthetic valve within the docking device 100, the docking device 100 may be further radially expanded, which can cause the coil 102 to rotate within the native annulus (also referred to as “clocking”). During the clocking, the slack 115 allows the inner cover 112 to be axially stretched and not rotate together with the coil 102 (that is, the coil 102 may slide axially relative to the inner cover 112). Because the guard member 104 can be fixedly attached to the retention member 114, the slack 115 can also prevent the guard member 104 from rotating and pinning open the native leaflets during the clocking.

In some examples, a portion of the inner cover 112 underlying the radially compressed petals 140 (e.g., from a portion between 6C-6C and 6D-6D to a portion marked by 110C in FIG. 6A) can have a smaller outer diameter than the remaining portion of the inner cover 112 and/or the retention member 114 so as to fit the guard member 104 (with the radially compressed petals 140) inside the dock sleeve of the delivery apparatus during delivery of the docking device 100 and after initial deployment of the docking device 100 at the implantation site. In some examples, the portion of the inner cover 112 underlying the radially compressed petals 140 can have an outer diameter that is between 1.10 mm and 1.40 mm (for example, about 1.20 mm), while the remaining portion of the inner cover 112 and/or the retention member 114 can have an outer diameter that is between 1.8 mm and 2.3 mm (for example, about 1.9 mm).

In various examples, the guard member 104 can help covering an atrial side of an atrioventricular valve to prevent and/or inhibit blood from leaking through the native leaflets, commissures, and/or around an outside of the prosthetic valve by blocking blood in the atrium from flowing in an atrial to ventricular direction (that is, antegrade blood flow)—other than through the prosthetic valve. Positioning the guard member 104 on the atrial side of the valve can additionally or alternatively help reduce blood in the ventricle from flowing in a ventricular to atrial direction (that is, retrograde blood flow).

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (that is, retrograde blood flow). Positioning the guard member 104 on the ventricular side of the valve can additionally or alternatively help reduce blood in the atrium from flowing in the atrial direction to ventricular direction (that is, antegrade blood flow)—other than through the prosthetic valve.

In some examples, the docking device 100 can include at least one radiopaque marker configured to provide visual indication about the location of the docking device 100 relative to its surrounding anatomy, and/or the amount of radial expansion of the docking device 100 (for example, when a prosthetic valve is subsequently deployed in the docking device 100) under fluoroscopy. For example, one or more radiopaque markers can be placed on the coil 102. In one particularly example, a radiopaque marker can be disposed at the central region 108 of the coil. In some examples, one or more radiopaque markers can be placed on the inner cover 112, the guard member 104, and/or other components of the docking device 100.

Additional examples and characteristics of the guard member are described in the section “Exemplary PVL Guards” below.

Additional examples of the docking device and its variants, including various examples of the coil, guard member, inner cover, and other components of the docking device, are described in International Publication No. WO/2020/247907, the entirety of which is incorporated by reference herein.

### Exemplary PVL Guards

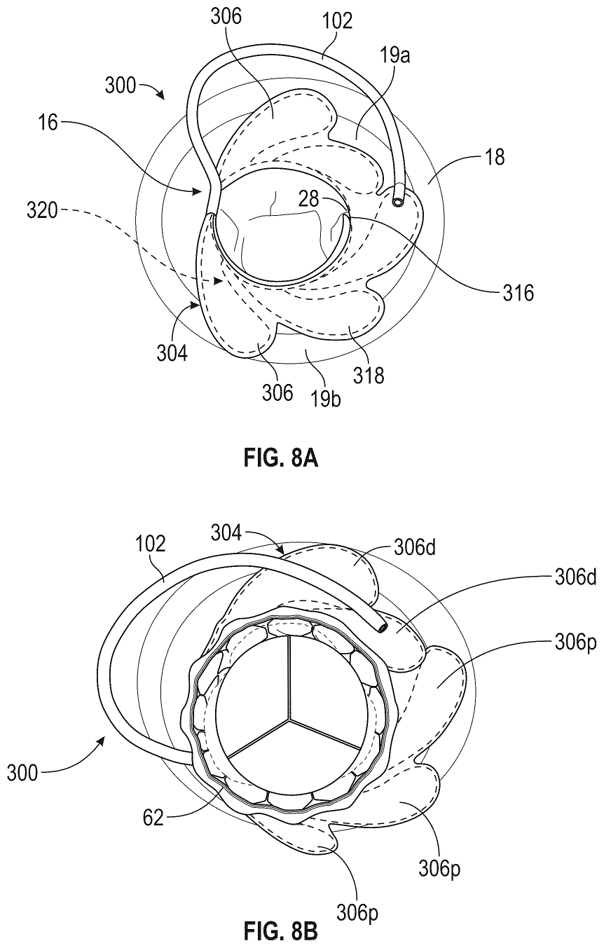

FIGS. 7A-7D depicts an example guard member 204. For a docking device, any of the guard members described herein can be interchangeable. For example, the guard member 204 can replace the guard member 104 of the docking device 100 described above.

The guard member 204 is movable between a radially compresses state and a radially expanded state. For example, FIGS. 7A-7B shows the guard member 204 in the radially expanded state, and FIGS. 7C-7D show the guard member 204 in the radially compressed state.

The guard member 204 is also movable between a curved state and a substantially straight state. For example, FIG. 7A shows the guard member 204 in the curved state and FIGS. 7B-7D show the guard member 204 in the substantially straight state.

The guard member 204 is also movable between a folded configuration and an unfolded configuration. For example, FIGS. 7A-7C show the guard member 204 in the unfolded configuration and FIG. 7D shows the guard member 204 in the folded configuration.

FIG. 7D depicts the guard member 204 in a delivery configuration, for example, when the guard member 204 is retained within a dock sleeve 55 of a delivery apparatus (for example, during delivery of the docking device and after initial deployment of the docking device at the implantation site). In the delivery configuration, the guard member 204 can be folded, radially compressed, and remain substantially straight. FIG. 7A depicts the guard member 204 in a deployed configuration, for example, after the docking device is deployed at the implantation site and the dock sleeve 55 is removed. In the deployed configuration, the guard member 204 can unfold, radially expand, and move to the curved state.

In the example depicted in FIGS. 7A-7D, the guard member 204 includes six petals 206, although the number of petals 206 can be more than six or less than six. The petals 206 can extend circumferentially along a portion of the coil 102. When the guard member 204 is in the radially compressed state (FIGS. 7C-7D), the petals 206 can be radially compressed against the coil 102. When the guard member 204 is in the radially expanded state, the petals 206 can extend radially outwardly relative to the coil 102.

Each petal 206 has a rounded head portion 208 and a tapered base portion 210. The head portion 208 can be wider than the base portion 210 when the guard member 204 is in the radially expanded state. For each petal 206, the base portion 210 can be attached to the coil 102 (for example, via sutures) and the head portion 208 can extend radially outwardly relative to the base portion 210.

The petals 206 can define an outer edge 212 and an inner edge 214 of the guard member 204. The guard member 204 can be attached to the coil 102 at the inner edge 214.

When the guard member 204 is in the curved state, the inner edge 214 can be curved with an arc angle A. In some examples, the arc angle A is greater than 180 degrees. In some examples, the arc angle A can be between 240 degrees and 360 degrees (for example, about 270 degrees), inclusive.

In some examples, when the guard member 204 is in the radially expanded state, each petal 206 can extend angularly relative to the coil 102 such that the outer edge 212 can have an undulating or scalloped shape like a rolling wave.

In some examples, the undulating outer edge 212 can have one or more petal junctions 215. Each petal junction 215 is located between two adjacent petals 206. In some examples, the one or more petal junctions 215 can be radially spaced apart from the coil 102 with about equal distance when the guard member 204 is in the radially expanded state.

The overall shape, size, and position of the petals 206 are configured to conform to the native anatomy of the implantation site (for example, the native mitral annulus) and do not puncture or erode through the adjacent native tissue. In some examples, when the guard member 204 is in the radially expanded state, the petals 206 can extend in the same angular direction (for example, clockwise or counterclockwise when viewed from the top or stabilization turn 110 of the docking device). For instance, in FIGS. 7A-7B, all six petals 206 extend in clockwise direction when viewed from above the figures. In other examples, at least two of the petals can extend in opposite angular directions (for example, one petal extends in the clockwise direction and another petal extends in the counterclockwise direction) when the guard member 204 is in the radially expanded state. For instance, in FIGS. 6A-6B, the distal petal 140d extends in clockwise direction while the three proximal petals 140p extend in counterclockwise direction when viewed from the top of the docking device 100.

In some examples, the number of petals in a guard member can be between three and eight, or between four and six, all inclusive. For example, FIGS. 7A-7D show that the guard member 204 has six petals 206, whereas FIGS. 6A-6B show that the guard member 104 has four petals 140.

In some examples, as depicted in FIGS. 7A-7B, the petals 206 have substantially the same size when the guard member 204 is in the radially expanded state. In other examples, at least two of petals 206 can have different sizes when the guard member 204 is in the radially expanded state.

In some examples, as depicted in FIGS. 7A-7B, the petals 206 have substantially the same shape when the guard member 204 is in the radially expanded state. In other examples, at least two of the petals 206 can have different shapes when the guard member 204 is in the radially expanded state.

As described above, the guard member 204 can move between a folded configuration and an unfolded configuration. For example, the guard member 204 can be folded at a hinge potion 216 which divides the plurality of petals 206 into one or more distal petals 206d (that is, the petals that are located distal to the hinge portion 216) and one or more proximal petals 206p (that is, the petals that are located proximal to the hinge portion 216).

In some examples, the number of distal petals can be less than the number of proximal petals. For example, the guard member 204 depicted in FIGS. 7B-7C has two distal petals 206d the four proximal petals 206p. In some examples, the number of distal petals can be the same as the number of proximal petals. In still other examples, the number of distal petals can be more than the number of proximal petals.

When the guard member 204 is in the radially compressed state, the distal petals can fold over and overlap with at least some of the proximal petals. For example, FIG. 7D shows two distal petals 206d folding over and overlapping with two of the proximal petals 206p. When the guard member 204 moves from the radially compressed state to the radially expanded state, the distal petals 206d can unfold and flip to an opposite side of the proximal petals 206p.

When deployed at a native heart valve, the distal petals 206d and the proximal petals 206p can be configured to press against opposing portions of a native heart chamber. For example, when deployed at the mitral valve, the distal petals 206d can be configured to press against an anterior leaflet of the mitral valve, and the proximal petals 206p can be configured to press against a posterior leaflet of the mitral valve. The hinge portion 216 can be positioned adjacent a medial commissure of the mitral valve.

The guard member 204 can include a wireframe 220 and a cover 218 substantially enclosing the wireframe 220. The shape of the wireframe 220 generally defines the shape of the guard member 204. For example, the wireframe 220 can include a spine 230 and a plurality of lobes 222 connected to the spine 230. The lobes 222 can extend radially outwardly from the spine 230. Each lobe 222 can extend along a periphery of a corresponding petal 206. The spine 230 can extend along the inner edge 214 of the guard member 204. The spine 230 can be curved to move the guard member 204 to the curved state or straightened to move the guard member 204 to the substantially straight state.

In some examples, the spine 230 and the lobes 222 are interconnected to form a unitary piece. For example, the spine 230 and the lobes 222 can be laser cut from a single sheet of metal or metal alloy. In other examples, the lobes 222 and the spine 230 can be created as separate components and then joined together (for example, via molding, welding, soldering, etc.) to form the wireframe 220.

The wireframe 220 can have the same states or configurations as the guard member 204. For example, the lobes 222 can be radially compressed or expanded as the guard member 204 moves between the radially compressed state and radially expanded state. The spine 230 can be curved or straightened as the guard member 204 moves between the curved state and substantially straight state. The spine 230 can also be folded or unfolded as the guard member 204 moves between the folded configuration and unfolded configuration.

Like the wireframe 120, the wireframe 220 can comprise a shape memory material, such as Nitinol. The wireframe 220 can be shape set so that the wireframe 220 is biased toward the deployed configuration. For example, when the guard member 204 is retained within a dock sleeve (for example, the dock sleeve 55) of a delivery apparatus during delivery of the docking device and after initial deployment of the docking device at the implantation site, the lobes 222 can be radially compressed and the spine 230 can be substantially straightened and folded. After deploying the docking device and removing the dock sleeve from the guard member 204, the spine 230 can unfold and become curved, and the lobes 222 can radially expand under the biasing force.

The cover 218 can be similar to the cover 118. For example, the cover 218 can be configured to be sufficiently elastic so that when the guard member 204 moves from the delivery configuration to the deployed configuration, the cover 218 can accommodate the wireframe 220 (for example, radial expansion of the lobes 222 can cause corresponding radial expansion of the cover 218). The cover 218 can also be configured to be atraumatic to native tissue and/or promote tissue ingrowth into the cover 218.

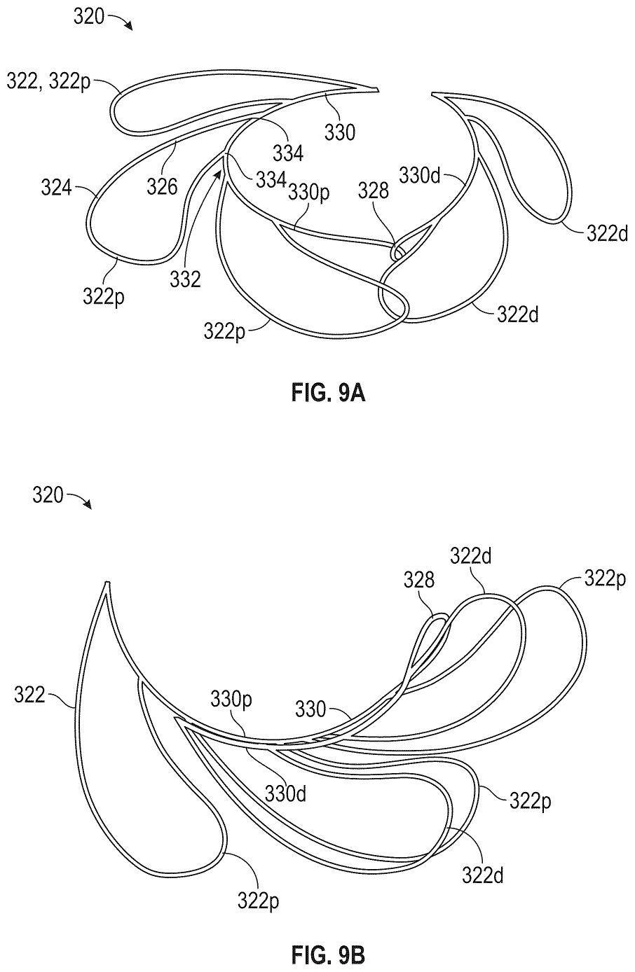

FIG. 8A depicts a docking device 300 deployed at a native valve, according to one example. FIG. 8B depicts the prosthetic valve 62 deployed within the docking device 300. The docking device 300 includes the coil 102 and a guard member 304 attached to the coil 102. In this example, the implantation site is the mitral valve 16 that separates the left atrium 18 from the left ventricle (the view is from the left atrial side and the left ventricle is behind the mitral annular plane).

In the depicted example, the guard member 304 includes five petals 306, including two distal petals 306d and three proximal petals 306p that are located on opposite sides of a hinge portion 316 of the guard member 304. In other examples, the guard member 304 can have different numbers of proximal and/or distal petals, and the total number of petals can be more or less than five.

Similarly, the guard member 304 can be retained within a dock sleeve (for example, the dock sleeve 55) and remain in a delivery configuration during delivery of the docking device 300 and after initial deployment of the docking device 300 at the native valve. In the delivery configuration, the guard member 304 can be substantially straight. The petals 306 can be radially compressed. Additionally, the distal petals 306d can be folded around the hinge portion 316 and overlap some of the proximal petals 306p. In some examples, the hinge portion 316 can comprise a loop 328, as described more fully below. The proximal petals 306p can be affixed to the coil 102 (for example, via sutures) and the distal petals 306d can be detached from the coil 102 to allow folding of the guard member 304 at the hinge portion 316.

After the docking device 300 is deployed at the native valve and the dock sleeve is removed, the guard member 304 can automatically move to a deployed configuration, as shown in FIGS. 8A-8B. In the deployed configuration, the guard member 304 can unfold itself, radially expand, and move to a curved state, as described above. As a result, the distal petals 306d can press against an anterior leaflet 19a of the mitral valve, and the proximal petals 306p can press against a posterior leaflet 19b of the mitral valve. Notably, in the deployed configuration, all petals 306 of the guard member 304 reside in the left atrium 18 while the coil 102 can extend into the left ventricle, for example, through a medial commissure 28 of the mitral valve. The petals 306 can act as flanges extending around the native mitral annulus, thereby preventing the docking device 300 from dropping into the left ventricle. Additionally, by keeping the guard member 304 in the left atrium 18, the risk of left ventricular outflow tract obstruction can also be reduced (for example, compared to otherwise extending the guard member into the left ventricle).

In some examples, the hinge portion 316 of the guard member 304 can be configured to align with the medial commissure 28 of the mitral valve when the guard member 304 is in the deployed configuration. One distal petal 306d and one proximal petal 306p that are immediately adjacent the hinge portion 316 can be configured to cover the area surrounding the medial commissure 28 so that paravalvular leakage at that location can be prevented or reduced.

Further, by pressing against opposing portions of the native mitral annulus, the guard member 304 can more securely anchor the docking device 300 at the mitral valve. For example, when radially expanding the prosthetic valve 62 within the docking device 300, the docking device 300 may be further radially expanded, which can cause clocking or rotation of the coil 102 within the mitral annulus. The rotation of the coil 102 may tend to move the docking device 300 toward the left ventricle. However, the hinge portion 316 can prevent the docking device 300 from dropping into the left ventricle due to clocking. As described above, the distal petals 306 stay in the left atrium 18 while the coil 102 can extend into the left ventricle. As such, a wedge can be formed at the hinge portion 316 which can catch the medial commissure 28 and prevent the docking device 300 from clocking into the left ventricle.

Similar to the guard members 104 and 204, the guard member 304 includes a wireframe 320 and a cover 318 substantially enclosing the wireframe 320. The cover 318 can be similar to the covers 118 and 218 described above.

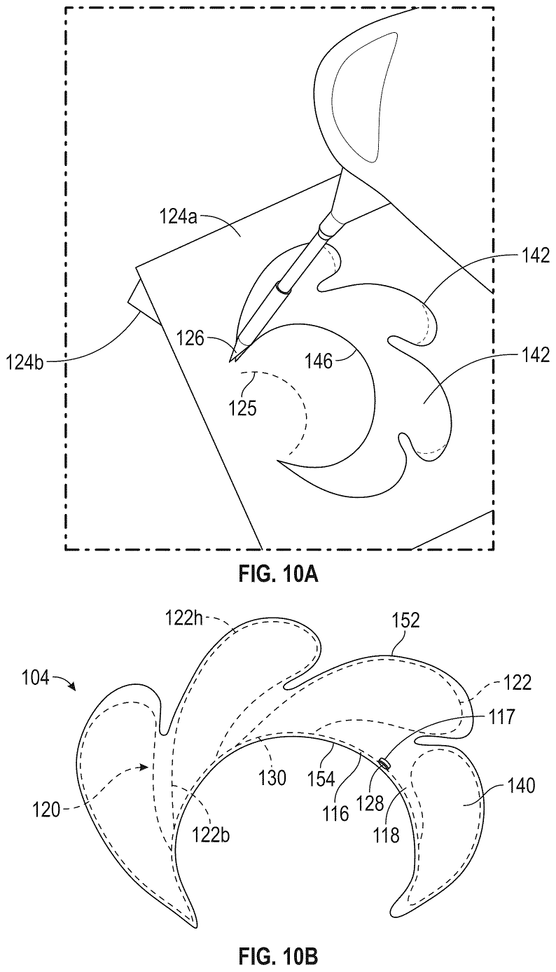

FIGS. 9A-9B show the wireframe 320 of the guard member 304. Like the wireframe 220, the wireframe 320 includes a spine 330 and a plurality of lobes 322 (five lobes are shown in this example) connected to the spine. The spine 330 can extend along an inner edge of the guard member 304, and each lobe 322 can extend along a periphery of a corresponding petal 306 of the guard member 304.

The lobes 322 can be radially expandable and compressible. As shown in FIG. 9A, each lobe 322 includes a head portion 324 and a base portion 326. For each lobe 322, the base portion 326 is narrower than the head portion 324 when the lobe 322 is radially expanded. The head and base portions of each lobe 322 generally define corresponding head and base portions of a petal 306 of the guard member 304.

In some examples, the base portion 326 of each lobe 322 can be connected to the spine 330 at two connecting points 334. In some examples, the base portions 326 of two adjacent lobes 322 can be separated by a gap 332 along the spine 330.

The spine 330 (and the wireframe 320) can be movable between a curved state (as shown in FIG. 9A) and a substantially straight state (similar to the spine 230 depicted in FIGS. 7B-7C). In some examples, when the spine 330 is in the curved state, the spine 330 can define an arc angle that is greater than 180 degrees.

The spine 330 (and the wireframe 320) can be movable between a folded configuration (as shown in FIG. 9B) and an unfolded configuration (as shown in FIG. 9A). For example, the spine 330 can have a proximal portion 330p, a distal portion 330d, and a loop 328 connecting the proximal portion 330p and the distal portion 330d. The loop 328 corresponds to the hinge portion 316 of the guard member 304, that is, the spine 330 can be folded at the loop 328. In some examples, the loop 328 can be configured as a coil spring which biases the spine 330 to move toward the unfolded configuration. To move the spine 330 to the folded configuration, an external force can be applied to overcome the biasing force of the loop 328.

The lobes 322 can include one or more distal lobes 322d connected to the distal portion 330d of the spine and one or more proximal lobes 322p connected to the proximal portion 330p of the spine (two distal lobes and three proximal lobes are shown in FIGS. 9A-9B). Thus, when the spine 330 is in the folded configuration, the distal lobes 322d can be folded relative to the proximal lobes 322p.

In the depicted example, when unfolded, the distal lobes 322d and the proximal lobes 322p extend in opposite angular direction. As a result, when folded, the distal lobes 322d can extend in the same angular direction as the proximal lobes 322p, and in some circumstances, can substantially overlap with some of the proximal lobes 322p. For example, FIG. 9B shows that the two distal lobes 322d substantially overlap with two of the proximal lobes 322p. In other examples, when unfolded, the distal lobes and the proximal lobes can extend in the same angular direction (like the lobes 222 depicted in FIG. 7D).

Because the loop 328 corresponds to the hinge portion 316 of the guard member 304, the loop 328 can align with the medial commissure 28 of the mitral valve when the guard member 304 is in the deployed configuration. In some examples, the cover 318 of the guard member 304 can have an opening or hole through which the loop 328 can extend out of the cover 318. In some examples, the hole can be positioned so that the loop 328 extends radially inwardly toward a central axis of the helical turns formed by the coil 102 (or the center of the native annulus) when deployed at the native valve. Such configuration can reduce the risk of tissue abrasion caused by the loop 328 exposed outside the cover 318. In some examples, the loop 328 extending out of the cover 318 can be enclosed within a protective member so as to further reduce the likelihood of abrasion to the surrounding tissue.

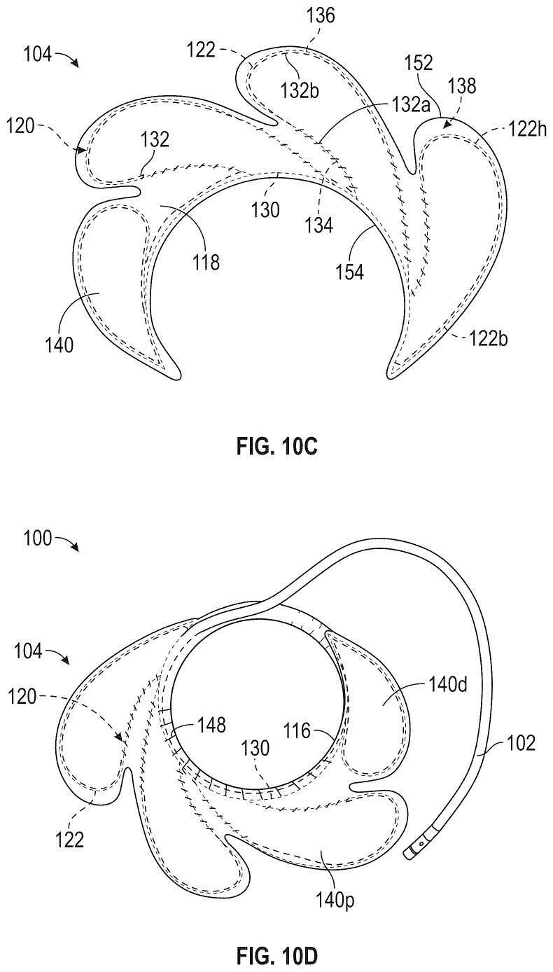

FIGS. 10A-10D illustrates an example method of making a docking device. In the depicted example, the docking device 100 having the guard member 104 is shown for illustration purposes, although it should be understood that similar method can be used to make docking devices having different guard members (for example, the guard members 204, 304, etc.).

In some examples, the wireframe 120 of the guard member 104 can be obtained by cutting a substrate to form the spine 130 and the lobes 122. Each lobe 122 has a head portion 122h and a base portion 122b. The base portions 122b are connected to the spine 130 of the wireframe 120. The head portions 122h are positioned farther away from the spine 130 than the base portions 122b. Each lobe 122 can have a tapered shape such that the head portion 122h is wider than the base portion. In one specific example, the wireframe 120 can be made by laser cutting a Nitinol sheet. In other examples, the lobes 122 and the spine 130 can be created as separate components and then joined together (for example, via molding, welding, soldering, etc.) to form the wireframe 120.

The guard member 104 can be created by enclosing the wireframe 120 into the cover 118. An example method of making the cover 118 is illustrated in FIGS. 10A-10B. In the depicted example, two fabric layers 124a, 124b can be stacked together.

The cover 118 can be created by cutting the two fabric layers 124a, 124b using a mold 142 placed over the two fabric layers 124a, 124b. The mold 142 matches the desired shape of the guard member 104 in the deployed configuration. For example, an outer periphery 144 of the mold 142 can define a shape of the lobes 122 of the wireframe 120. An inner periphery 146 of the mold 142 can defines a shape of the spine 130 of the wireframe 120.

In some examples, the two fabric layers 124a, 124b can be cut following a specific sequence. First, a heated soldering iron 126 can be moved along the outer periphery 144 of the mold 142 so that the two fabric layers 124a, 124b are heat-cut along the outer periphery 144 of the mold and sealed together to form an outer edge 152 of the cover 118. The outer edge 152 of the cover 118 defines the undulating or scalloped shape of the petals 140. Next, the wireframe 120 can be inserted between the two fabric layers 124a, 124b, for example, through an opening (marked by the dashed line 125) which can be created at a location that is radially inwardly of the inner periphery 146 of the mold 142. The wireframe 120 inserted between the two fabric layers 124a, 124b can be positioned so that the lobes 122 are aligned with the outer edge 152 of the cover 118. As such, each lobe 122 is retained within a corresponding petal 140. Then, the heated soldering iron 126 can be moved along the inner periphery 146 of the mold 142 so that the two fabric layers 124a, 124b are heat-cut along the inner periphery 146 the mold 142 and sealed together to form an inner edge 154 of the cover 118. As a result, the spine 130 of the wireframe 120 extends along the inner edge 154 of the cover 118.

In other examples, the cover 118 can be created by different methods. For example, instead of using the heated soldering iron 126, the two fabric layers 124a, 124b can be cut using scissors, laser beams, or other cutting methods. After the cutting, the inner edge 154 and/or outer edge 152 of the cover 118 can be sealed by heat, sutures, adhesives, or other sealing methods.

In some examples, the wireframe 120 can be connected to the cover 118 via a plurality of sutures 132. As shown in FIG. 10C, the sutures 132 can run through specific patterns so as to retain the wireframe 120 while also allow certain movability of the wireframe 120 within the cover 118.

For example, at least some of the sutures 132, referred to as cross-sutures 132a, can extend across one or more wire segments 134 located at base portions 122b of the lobes of the wireframe. Such cross-sutures 132a can retain each lobe 122 within its corresponding petal 140 and restrict lateral movement of the wireframe 120 within the cover 118.

Additionally, at least some of the sutures 132, referred to as inner sutures 132b, can extend along and located inwardly of one or more wire segments 136 located at head portions 122h of the lobes 122 of the wireframe. As a results, pockets 138 can be created between the line of inner sutures 132b and the outer edge 152 of the cover 118. The pockets 138 allow limited sliding movement of the lobes 122 within the cover 118. For example, the head portions 122h of the lobes can slide within the pockets 138, thereby allowing the wireframe 120 to move between the radially compressed state and radially expanded state, and/or between the substantially straight state and curved state. Notably, the cross-sutures 132a do not hinder sliding movement of the lobes 122 within the cover 118.

As shown in FIG. 10D, the guard member 104 can be attached to the coil 102 of the docking device 100. For example, the cover 118 and/or the wireframe 120 of the guard member 104 can be attached to the coil 102 via one or more sutures 148.

Before implanting the docking device 100, the guard member 104 can be retained within a dock sleeve (for example, the dock sleeve 55). The guard member 104 retained within the dock sleeve can remain in a radially compressed state. For example, the petals 140 can be radially compressed so that they extend along and are substantially parallel to the coil 102.

The guard member 104 retained within the dock sleeve can also be maintained in a folded configuration. For example, the distal petal 140d can be folded over relative to the proximal petals 140p at a hinge portion 116 therebetween. In some examples, the hinge portion 116 can include a loop 128 (similar to the loop 328) formed at the spine 130 of the wireframe.

As shown in FIG. 10B, a hole 117 can be created on the cover 118, through which the loop 128 can be exposed and extend out of the cover 118. In some examples, the loop 128 extending out of the cover 118 can be covered with a protective layer to reduce the likelihood of abrasion to the surrounding tissue. In some examples, the protective layer can include a fabric layer, a polymeric layer, or one or more suture loops.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking device for securing a prosthetic valve at a native valve, the docking device comprising: a coil comprising a plurality of helical turns when deployed at the native valve; and a guard member attached to the coil and is movable between a radially compressed state and a radially expanded state, wherein the guard member comprises a plurality of petals, wherein each petal comprises a rounded head portion and a tapered base portion, wherein the head portion is wider than the base portion when the guard member is in the radially expanded state.

Example 2. The docking device of any example herein, particularly example 1, wherein when the guard member is in the radially compressed state, the plurality of petals are radially compressed against the coil so that a radial profile of the docking device is smaller than a predefined threshold.

Example 3. The docking device of any example herein, particularly example 2, wherein the predefined threshold is between 2 mm and 3 mm, inclusive.

Example 4. The docking device of any example herein, particularly any one of examples 1-3, wherein when the guard member moves from the radially compressed state to the radially expanded state, the plurality of petals extend radially outwardly relative to the coil.

Example 5. The docking device of any example herein, particularly any one of examples 1-4, wherein when the guard member is in the radially expanded state, the plurality of petals extend circumferentially along a portion of the coil.

Example 6. The docking device of any example herein, particularly example 5, wherein the plurality of petals define an inner edge which has an arc angle that is greater than 180 degrees.

Example 7. The docking device of any example herein, particularly example 6, wherein the arc angle is between 240 degrees and 360 degrees, inclusive.

Example 8. The docking device of any example herein, particularly any one of examples 1-7, wherein the number of petals is between three and eight, inclusive.

Example 9. The docking device of any example herein, particularly example 8, wherein the number of petals is between four and six, inclusive.

Example 10. The docking device of any example herein, particularly any one of examples 1-9, wherein the plurality of petals have substantially the same size when the guard member is in the radially expanded state.

Example 11. The docking device of any example herein, particularly any one of examples 1-9, wherein at least two of the plurality of petals have different sizes when the guard member is in the radially expanded state.

Example 12. The docking device of any example herein, particularly any one of examples 1-11, wherein the plurality of petals have substantially the same shape when the guard member is in the radially expanded state.

Example 13. The docking device of any example herein, particularly any one of examples 1-11, wherein at least two of the plurality of petals have different shapes when the guard member is in the radially expanded state.

Example 14. The docking device of any example herein, particularly any one of examples 1-13, wherein for each petal, the base portion is attached to the coil and the head portion extends radially outwardly relative to the base portion when the guard member is in the radially expanded state.

Example 15. The docking device of any example herein, particularly any one of examples 1-14, wherein each petal extends angularly relative to the coil when the guard member is in the radially expanded state.

Example 16. The docking device of any example herein, particularly example 15, wherein the plurality of petals extend in the same angular direction when the guard member is in the radially expanded state.

Example 17. The docking device of any example herein, particularly example 15, wherein at least two of the plurality of petals extend in opposite angular directions when the guard member is in the radially expanded state.

Example 18. The docking device of any example herein, particularly any one of examples 1-17, wherein the plurality of petals define an undulating outer edge comprising one or more petal junctions, wherein each petal junction is located between two adjacent petals.

Example 19. The docking device of any example herein, particularly example 18, wherein the one or more petal junctions are radially spaced apart from the coil with about equal distance when the guard member is in the radially expanded state.

Example 20. The docking device of any example herein, particularly any one of examples 1-19, wherein the plurality of petals are connected to the coil via one or more sutures.

Example 21. The docking device of any example herein, particularly any one of examples 1-20, wherein the plurality of petals comprises one or more distal petals and one or more proximal petals, wherein the one or more proximal petals are connected to the one or more proximal petals at a hinge portion of the guard member.

Example 22. The docking device of any example herein, particularly example 21, wherein when the guard member is in the radially compressed state, the one or more distal petals fold over and overlap with at least some of the proximal petals.

Example 23. The docking device of any example herein, particularly example 22, when the guard member moves from the radially compressed state to the radially expanded state, the one or more distal petals unfold and flip to an opposite side of the one or more proximal petals.

Example 24. The docking device of any example herein, particularly any one of examples 21-23, wherein the number of distal petals is the same as the number of proximal petals.

Example 25. The docking device of any example herein, particularly any one of examples 21-23, wherein the number of distal petals is less than the number of proximal petals.

Example 26. The docking device of any example herein, particularly any one of examples 21-25, wherein when the coil is deployed at the native valve, the one or more distal petals press against a first portion of a native heart chamber and the one or more proximal petals press against a second portion of the native heart chamber that is opposite to the first portion.

Example 27. The docking device of any example herein, particularly example 26, wherein the native valve is a mitral valve, wherein the first portion comprises an anterior leaflet of the mitral valve, and the second portion comprises a posterior leaflet of the mitral valve, wherein the hinge portion of the guard member is positioned adjacent a medial commissure of the mitral valve.

Example 28. The docking device of any example herein, particularly any one of examples 21-27, wherein the one or more proximal petals are fixedly attached to the coil, and wherein the one or more distal petals are detached from the coil.

Example 29. The docking device of any example herein, particularly any one of examples 21-28, wherein the guard member comprises a wireframe and a cover substantially enclosing the wireframe.

Example 30. The docking device of any example herein, particularly example 29, wherein the wireframe comprises a loop at the hinge portion.

Example 31. The docking device of any example herein, particularly any one of examples 29-30, wherein the wireframe comprises a shape memory material.

Example 32. The docking device of any example herein, particularly example 31, wherein the shape memory material comprises nickel titanium alloy.

Example 33. The docking device of any example herein, particularly any one of examples 29-32, wherein the wireframe comprises a spine and a plurality of lobes connected to the spine, wherein the lobes extend radially outwardly from the spine.

Example 34. The docking device of any example herein, particularly example 33, wherein the spine and the plurality of lobes are interconnected to form a unitary piece.

Example 35. The docking device of any example herein, particularly any one of examples 33-34, wherein each lobe extends along a periphery of a corresponding petal.

Example 36. The docking device of any example herein, particularly any one of examples 33-35, wherein each lobe is connected to the spine at two connecting points that are axially spaced apart from one another.

Example 37. The docking device of any example herein, particularly any one of examples 33-36, wherein two adjacent lobes are axially separated by a gap along the spine.

Example 38. The docking device of any example herein, particularly any one of examples 30-37, wherein the loop extends out of the cover through a hole on the cover.

Example 39. The docking device of any example herein, particularly example 38, wherein the hole is positioned so that the loop extends radially inwardly toward a central axis of the helical turns formed by the coil when deployed at the native valve.

Example 40. The docking device of any example herein, particularly any one of examples 30-39, wherein the loop is enclosed within a protective member.

Example 41. A docking device for securing a prosthetic valve at a native valve, the docking device comprising: a coil comprising a plurality of helical turns when deployed at the native valve; and a guard member attached to the coil and is movable between a radially compressed state and a radially expanded state, wherein the guard member comprises a wireframe including a spine and a plurality of lobes connected to the spine, wherein the lobes extend radially outwardly from the spine.

Example 42. The docking device of any example herein, particularly example 41, wherein the guard member further comprises a cover substantially enclosing the wireframe.

Example 43. The docking device of any example herein, particularly any one of examples 41-42, wherein each lobe comprises a rounded head portion and a tapered base portion, wherein the head portion is wider than the base portion when the guard member is in the radially expanded state.

Example 44. The docking device of any example herein, particularly example 43, wherein the plurality of lobes are radially expandable and compressible.

Example 45. The docking device of any example herein, particularly any one of examples 41-44, wherein the plurality of lobes comprise one or more proximal lobes and one or more distal lobes, wherein the one or more distal lobes are configured to be foldable relative to the one or more proximal lobes.

Example 46. The docking device of any example herein, particularly example 45, wherein the one or more distal lobes fold over the one or more proximal lobes when the guard member is in the radially compressed state, wherein the one or more distal lobes are unfolded from the one or more proximal lobes when the guard member is in the radially expanded state.

Example 47. The docking device of any example herein, particularly any one of examples 45-46, wherein the wireframe comprises a loop located between the one or more proximal lobes and the one or more distal lobes.

Example 48. The docking device of any example herein, particularly any one of examples 45-47, wherein when unfolded, the one or more distal lobes and the one or more proximal lobes extend in the same angular direction.

Example 49. The docking device of any example herein, particularly any one of examples 45-47, wherein when unfolded, the one or more distal lobes and the one or more proximal lobes extend in opposite angular direction.

Example 50. The docking device of any example herein, particularly any one of examples 41-49, wherein when the guard member is in the radially expanded state, the spine defines an arc angle that is greater than 180 degrees.

Example 51. A guard member for a docking device configured to secure a prosthetic valve at a native valve, the guard member comprising: a wireframe; and a cover substantially enclosing the wireframe, wherein the wireframe comprises a spine and a plurality of lobes connected to the spine, wherein the lobes extend radially outwardly from the spine, wherein the plurality of lobes are radially expandable and compressible.

Example 52. The guard member of any example herein, particularly example 51,wherein each lobe comprises a head portion and a base portion, wherein the base portion is narrower than the head portion when the lobe is radially expanded.

Example 53. The guard member of any example herein, particularly example 52,wherein the base portion of each lobe is connected to the spine at two connecting points.

Example 54. The guard member of any example herein, particularly any one of examples 52-53, wherein the base portions of two adjacent lobes are separated by a gap along the spine.

Example 55. The guard member of any example herein, particularly any one of examples 51-54, wherein the spine is movable between a folded configuration and an unfolded configuration.

Example 56. The guard member of any example herein, particularly any one of examples 51-55, wherein the spine is movable between a curved state and a substantially straight state.

Example 57. The guard member of any example herein, particularly any one of examples 55-56, wherein the plurality of lobes comprise one or more proximal lobes and one or more distal lobes, wherein the one or more distal lobes are folded over the one or more proximal lobes when the spine is in the folded configuration, and wherein the one or more distal lobes are unfolded from the one or more proximal lobes when the spine is in the unfolded configuration.

Example 58. The guard member of any example herein, particularly example 57, wherein the spine comprises a hinge portion where the one or more distal lobes can fold relative to the one or more proximal lobes.

Example 59. The guard member of any example herein, particularly example 58, wherein the spine comprises a proximal portion and a distal portion connected by a loop at the hinge portion.

Example 60. The guard member of any example herein, particularly example 59, wherein the loop is configured to bias the spine to move from the folded configuration to the unfolded configuration.

Example 61. A method for making a docking device configured to secure a prosthetic valve at a native valve, the method comprising: obtaining a wireframe comprising a spine and a plurality of lobes connected to and extending radially outwardly from the spine; and enclosing the wireframe with a cover to form a guard member of the docking device.

Example 62. The method of any example herein, particularly example 61, wherein the obtaining the wireframe comprises cutting a substrate to form the spine and the plurality of lobes.

Example 63. The method of any example herein, particularly example 62, wherein the substrate comprises a Nitinol sheet, and wherein the cutting comprises laser cutting the Nitinol sheet.

Example 64. The method of any example herein, particularly any one of examples 62-63, further comprising stacking two fabric layers together and cutting the two fabric layers using a mold placed over the two fabric layers to create the cover, wherein an outer periphery of the mold defines a shape of the plurality of lobes of the wireframe, and wherein an inner periphery of the mold defines a shape of the spine of the wireframe.

Example 65. The method of any example herein, particularly example 64, wherein cutting the two fabric layers comprises moving a heated soldering iron along the outer periphery of the mold so that the two fabric layers are heat-cut along the outer periphery of the mold and sealed together to form an outer edge of the cover.

Example 66. The method of any example herein, particularly example 65, wherein enclosing the wireframe comprises inserting the wireframe between the two fabric layers through an opening that is located radially inwardly of the inner periphery of the mold, wherein the wireframe inserted between the two fabric layers is positioned so that the plurality of lobes are aligned with the outer edge of the cover.

Example 67. The method of any example herein, particularly example 66, wherein enclosing the wireframe further comprises moving the heated soldering iron along the inner periphery of the mold so that the two fabric layers are heat-cut along the inner periphery the mold and sealed together to form an inner edge of the cover, wherein the spine of the wireframe extends along the inner edge of the cover.

Example 68. The method of any example herein, particularly any one of examples 61-66, further comprising connecting the wireframe to the cover via a plurality of sutures.

Example 69. The method of any example herein, particularly example 68, wherein at least some of the sutures extend across one or more wire segments located at base portions of the plurality of lobes of the wireframe, wherein the base portions are connected to the spine of the wireframe.

Example 70. The method of any example herein, particularly any one of examples 68-69, wherein at least some of the sutures extend along and located inwardly of one or more wire segments located at head portions of the plurality of lobes of the wireframe, wherein the head portions are positioned farther away from the spine than the base portions.

Example 71. The method of any example herein, particularly any one of examples 61-70, further comprising attaching the guard member to a coil of the docking device.

Example 72. The method of any example herein, particularly example 71, wherein the attaching comprises suturing the cover or the wireframe to the coil of the docking device.

Example 73. The method of any example herein, particularly any one of examples 61-72, further comprising folding the guard member at a hinge portion of the spine, wherein the folding causes one or more lobes located at a distal end portion of the spine to fold over one or more lobes located at a proximal end portion of the spine.

Example 74. The method of any example herein, particularly example 73, further comprising radially compressing the guard member so that the guard member can be retained within a dock sleeve.

Example 75. The method of any example herein, particularly any one of examples 73-74, further comprising creating a hole on the cover, and exposing the hinge portion of the spine through the hole.

Example 76. The method of any example herein, particularly example 75, further comprising covering the hinge portion with a protective layer.

Example 77. The method of any example herein, particularly example 76, wherein the protective layer comprises a fabric layer, a polymeric layer, or one or more suture loops.

Example 78. A method comprising: delivering a docking device to a native valve; deploying the docking device at an annulus of the native valve; and deploying a prosthetic valve within the docking device, wherein the docking device comprises a coil and a guard member attached to the coil, wherein the coil remains in a substantially straight configuration when delivering the docking device and moves to a helical configuration after the docking device is deployed, wherein the guard member remains in a folded configuration when delivering the docking device and moves to an unfolded configuration after the docking device is deployed.

Example 79. The method of any example herein, particularly example 78, wherein delivering the docking device comprises retaining the docking device within a dock sleeve, wherein deploying the docking device comprises moving the docking device out of the dock sleeve.

Example 80. The method of any example herein, particularly any one of examples 78-79, wherein deploying the docking device comprises aligning a hinge portion of the guard member with a medial commissure of the native valve, wherein the guard member folds or unfolds around the hinge portion.

Example 81. A method comprising sterilizing the docking device or guard member of any example herein, particularly any one of examples 1-60.

Example 82. A method of treating a heart on a simulation, the method comprising: deploying a docking device at a target location; and deploying a prosthetic valve within the docking device; wherein the docking device is according to any one of examples 1-50.

The features described herein with regard to any example can be combined with other features described in any one or more of the other examples, unless otherwise stated. For example, any one or more of the features of one docking device can be combined with any one or more features of another docking device.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A docking device for securing a prosthetic valve at a native valve, the docking device comprising:
a coil comprising a plurality of helical turns when deployed at the native valve; and
a guard member attached to the coil and is movable between a radially compressed state and a radially expanded state,
wherein the guard member comprises a plurality of petals, wherein each petal comprises a rounded head portion and a tapered base portion, wherein the head portion is wider than the base portion when the guard member is in the radially expanded state.

2. The docking device of claim 1, wherein when the guard member is in the radially compressed state, the plurality of petals is radially compressed against the coil so that a radial profile of the docking device is smaller than a predefined threshold.

3. The docking device of claim 1, wherein when the guard member moves from the radially compressed state to the radially expanded state, the plurality of petals extend radially outwardly relative to the coil.

4. The docking device of claim 1, wherein when the guard member is in the radially expanded state, the plurality of petals extends circumferentially along a portion of the coil.

5. The docking device of claim 4, wherein the plurality of petals defines an inner edge which has an arc angle that is greater than 180 degrees.

6. The docking device of claim 1, wherein for each petal, the base portion is attached to the coil and the head portion extends radially outwardly relative to the base portion when the guard member is in the radially expanded state.

7. The docking device of claim 1, wherein each petal extends angularly relative to the coil when the guard member is in the radially expanded state.

8. The docking device of claim 7, wherein the plurality of petals extends in the same angular direction when the guard member is in the radially expanded state.

9. The docking device of claim 7, wherein at least two of the plurality of petals extend in opposite angular directions when the guard member is in the radially expanded state.

10. The docking device of claim 1, wherein the plurality of petals defines an undulating outer edge comprising one or more petal junctions, wherein each petal junction is located between two adjacent petals.

11. The docking device of claim 1, wherein the plurality of petals is connected to the coil via one or more sutures.

12. The docking device of claim 1, wherein the plurality of petals comprises one or more distal petals and one or more proximal petals, wherein the one or more proximal petals are connected to the one or more proximal petals at a hinge portion of the guard member.

13. A docking device for securing a prosthetic valve at a native valve, the docking device comprising:
a coil comprising a plurality of helical turns when deployed at the native valve; and
a guard member attached to the coil and is movable between a radially compressed state and a radially expanded state,
wherein the guard member comprises a wireframe including a spine and a plurality of lobes connected to the spine, wherein the lobes extend radially outwardly from the spine.

14. The docking device of claim 13, wherein the guard member further comprises a cover substantially enclosing the wireframe.

15. The docking device of claim 13, wherein each lobe comprises a rounded head portion and a tapered base portion, wherein the head portion is wider than the base portion when the guard member is in the radially expanded state.

16. The docking device of claim 15, wherein the plurality of lobes is radially expandable and compressible.

17. The docking device of claim 13, wherein the plurality of lobes comprises one or more proximal lobes and one or more distal lobes, wherein the one or more distal lobes are configured to be foldable relative to the one or more proximal lobes.

18. The docking device of claim 17, wherein the one or more distal lobes fold over the one or more proximal lobes when the guard member is in the radially compressed state, wherein the one or more distal lobes are unfolded from the one or more proximal lobes when the guard member is in the radially expanded state.

19. The docking device of claim 17, wherein the wireframe comprises a loop located between the one or more proximal lobes and the one or more distal lobes.

20. A method comprising:
delivering a docking device to a native valve;
deploying the docking device at an annulus of the native valve; and
deploying a prosthetic valve within the docking device,
wherein the docking device comprises a coil and a guard member attached to the coil,
wherein the coil remains in a substantially straight configuration when delivering the docking device and moves to a helical configuration after the docking device is deployed,
wherein the guard member remains in a folded configuration when delivering the docking device and moves to an unfolded configuration after the docking device is deployed.
